(19) United States
(12) Patent Application Publication
(10) Pub. No.: US 2025/0166816 A1
(43) Pub. Date: May 22, 2025

(54) MEASUREMENT DEVICE, INFORMATION TERMINAL, AND OPERATION RECORDING MEDIUM FOR INFORMATION TERMINAL

(71) Applicant: OMRON HEALTHCARE Co., Ltd., Kyoto (JP)

(72) Inventor: Nozomi NAKAWAKI, Kyoto (JP)

(21) Appl. No.: 19/032,335

(22) Filed: Jan. 20, 2025

Related U.S. Application Data
(63) Continuation of application No. PCT/JP2023/018461, filed on May 17, 2023.

(30) Foreign Application Priority Data
Sep. 21, 2022 (JP) 2022-150402

(51) Int. Cl. G16H 40/63; H04W 4/80

(52) U.S. Cl. CPC G16H 40/63; H04W 4/80

(57) ABSTRACT

A measurement device, an information terminal, and an operation recording medium of the information terminal that can perform pairing of the information terminal and the measurement device easily and safely. A communication connection between a blood pressure monitor and a smartphone is established by near-field wireless communication, and in this state, operation information indicating a specific operation for operating a sensor unit included in the blood pressure monitor is transmitted from the smartphone to the blood pressure monitor, and information instructing the specific operation is displayed on a display unit of the smartphone, and encryption information is shared between the blood pressure monitor and the smartphone when the specific operation is performed within a predetermined time after the operation information is transmitted from the smartphone to the blood pressure monitor.

### CROSS-REFERENCE TO RELATED APPLICATIONS

This application is the U.S. national stage application filed pursuant to 35 U.S.C. 365(c) and 120 as a continuation of International Patent Application No. PCT/JP2023/018461, filed May 17, 2023, which application claims priority to Japanese Patent Application No. 2022-150402, filed Sep. 21, 2022, which applications are incorporated herein by reference in their entireties.

### TECHNICAL FIELD

The present invention relates to a measurement device, an information terminal, and an operation recording medium for an information terminal.

### BACKGROUND ART

The measurement device described in the present specification includes a biological information measurement device that measures biological information such as a weight, a body composition, a blood pressure, a pulse, a heart rate, a body temperature, blood glucose, or a blood oxygen saturation level, and an activity amount measurement device that measures an activity amount such as the number of steps, a walking distance, or calorie consumption. The measurement device includes a measurement sensor for measuring a measurement target amount. The measurement target amount of the measurement sensor includes biological information such as a weight, a body fat percentage, a blood pressure value, a pulse rate, a heart rate, a body temperature, a blood glucose level, or a blood oxygen saturation level, and an activity amount such as the number of steps, a walking distance, or calorie consumption, depending on the measurement device. A measurement result of such a measurement device is recorded and analyzed by an information terminal such as a smartphone, a tablet terminal, a notebook personal computer, and a desktop personal computer.

In the case of recording and analyzing such a measurement result, it is desirable that the measurement device and the information terminal are communicably connected to each other so that the information terminal can automatically acquire the measurement result, instead of the user inputting the measurement result to the information terminal each time. Specifically, for example, a method in which the information terminal receives the measurement result from the measurement device by near-field wireless communication such as Bluetooth (trade name) is conceivable.

Patent Literature 1 describes a method for pairing a measurement instrument and an information terminal. The pairing method includes a measurement step of obtaining a predetermined measurement target amount in a measurement instrument; a first display step of displaying the measurement target amount on the measurement instrument; a transmission step of transmitting measurement information including identification information for identifying the measurement instrument and the measurement target amount from the measurement instrument; a receiving step of receiving, by an information terminal, the measurement information; a second display step of displaying a value of the measurement target amount included in the received measurement information on the information terminal; an input step of receiving an input as to whether or not a value of the measurement target amount displayed on the measurement instrument and a value of the measurement target amount displayed on the information terminal match each other; and a storage step of storing, in the information terminal, the identification information of the measurement instrument that has transmitted the measurement information including the value of the measurement target amount when there is an input indicating that the value of the measurement target amount displayed on the measurement instrument matches the value of the measurement target amount displayed on the information terminal in the input step.

Patent Literature 2 describes a wireless communication device that measures a radio wave strength of a received radio wave from an information processing device and does not have an input/output interface, the wireless communication device including a request unit that requests the information processing device to present, to a user of the wireless communication device, a message requesting the user to perform one of a first operation that causes a change in the radio wave strength of the received radio wave measured by the wireless communication device and a second operation that does not cause a change in the radio wave strength of the received radio wave measured by the wireless communication device; and a selection unit that detects whether or not there is a change in the radio wave strength of the received radio wave during a time limit predetermined after the request unit requests the information processing device to present the message, selects one value of logical values in a case where there is a change in the radio wave strength of the received radio wave during the time limit, and selects the other value of the logical values in a case where there is no change in the radio wave strength of the received radio wave during the time limit.

### CITATION LIST-PATENT LITERATURE

Patent Literature 1: JP 2020-098467 A

Patent Literature 2: WO 2017/187567

### SUMMARY OF INVENTION

### Technical Problem

In order to perform secure near-field wireless communication between a measurement device and an information terminal, processing of pairing the measurement device and the information terminal is necessary. The pairing refers to processing of exchanging encryption information used for near-field wireless communication between the measurement device and the information terminal and registering the information with each other. When a special operation is required for the pairing, a user who is not good at operating the instrument cannot complete the pairing, and cannot manage the measurement result of the measurement device by the information terminal. In addition, if the communication between the measurement device and the information terminal is established and the pairing is completed only by a simple operation of the information terminal (for example, an operation of permitting the pairing, or the like), the pairing can be performed between the information terminal irrelevant to the user and the measurement device, which causes a concern about safety.

An object of the present invention is to provide a measurement device, an information terminal, and an operation recording medium of an information terminal that can perform pairing of the information terminal and the measurement device easily and safely.

### Solution To Problem

The above problems can be solved by the following configurations. Note that corresponding components and the like in the following embodiments are illustrated in parentheses, but the embodiments are not limited thereto.

(1) A measurement device (blood pressure monitor 10) including a near-field wireless communication unit (communication unit 12), a processor (processor 11), and a measurement sensor (sensor unit 16), wherein the processor receives, in a state in which a communication connection with an information terminal by near-field wireless communication is established, operation information indicating a specific operation for operating the measurement sensor from the information terminal (smartphone 20); and shares, with the information terminal and in a case where the specific operation is determined to be performed within a predetermined time (upper limit time) after reception of the operation information, encryption information for performing encrypted communication by the near-field wireless communication.

According to the configuration of (1), the user can complete the pairing only by establishing the connection between the measurement device and the information terminal and operating, in this state, the measurement device in accordance with the normal purpose of use, for example, in accordance with the operation information output from the information terminal. Therefore, even a user who is not good at operating the instrument can complete pairing without hesitation, and can start cooperation between the measurement device and the information terminal. In addition, since pairing is not completed unless a specific operation is performed within a predetermined time, it is possible to prevent pairing with an unintended information terminal from being completed.

(2) The measurement device according to (1), wherein the measurement device measures biological information or an activity amount, and the specific operation includes an operation of measuring the biological information or an operation of moving the measurement device.

According to the configuration of (2), the pairing can be completed only by starting the measurement of the biological information or moving the measurement device, and even a user who is not good at operating the instrument can complete pairing without hesitation.

(3) The measurement device according to (1) or (2), wherein the processor receives the operation information from the information terminal at a plurality of timings, and shares the encryption information with the information terminal in a case where the specific operation is determined to be performed within a predetermined time after reception of each piece of the operation information.

According to the configuration of (3), the pairing can be completed when a specific operation is executed a plurality of times. By increasing the number of operations required to complete pairing, it is possible to securely prevent pairing with an unintended information terminal from being completed.

(4) The measurement device according to (3), wherein a plurality of pieces of the operation information received at the plurality of timings include information indicating a first operation as the specific operation and information indicating a second operation as the specific operation, the second operation being different from the first operation.

According to the configuration of (4), the pairing can be completed when a plurality of types of operations are executed. By increasing the types of operations for completing pairing, it is possible to securely prevent pairing with an unintended information terminal from being completed.

(5) The measurement device according to (4), wherein the measurement device measures an activity amount, the first operation is an operation of moving the measurement device, and the second operation is an operation of staying the measurement device.

According to the configuration of (5), the pairing can be completed by moving and staying the measurement device. By increasing the types of operations for completing pairing, it is possible to securely prevent pairing with an unintended information terminal from being completed.

(6) An information terminal (smartphone 20) including a near-field wireless communication unit (communication unit 22) and a processor (processor 21), wherein the processor transmits, to a measurement device (blood pressure monitor 10) and in a state in which a communication connection with the measurement device by near-field wireless communication is established, operation information indicating a specific operation for operating a measurement sensor (sensor unit 16) included in the measurement device and outputs information instructing the specific operation, and shares, with the measurement device and in a case where the specific operation is determined to be performed within a predetermined time (upper limit time) after transmission of the operation information, encryption information for performing encrypted communication by the near-field wireless communication.

(7) An operation recording medium of an information terminal (smartphone 20) including a near-field wireless communication unit (communication unit 22), the operation recording medium causing a processor (processor 21) to execute transmitting, to a measurement device (blood pressure monitor 10) and in a state in which a communication connection with the measurement device by near-field wireless communication is established, operation information indicating a specific operation for operating a measurement sensor (sensor unit 16) included in the measurement device and outputting information instructing the specific operation, and sharing, with the measurement device and in a case where the specific operation is determined to be performed within a predetermined time (upper limit time) after transmission of the operation information, encryption information for performing encrypted communication by the near-field wireless communication.

In other aspects, the present disclosure also relates to a measurement device, an information terminal, and an operation recording medium of the information terminal that can perform pairing of the information terminal and the measurement device easily and safely. A communication connection between a blood pressure monitor (10) and a smartphone (20) is established by near-field wireless communication, and in this state, operation information indicating a specific operation for operating a sensor unit (16) included in the blood pressure monitor (10) is transmitted from the smartphone (20) to the blood pressure monitor (10), and information instructing the specific operation is displayed on a display unit (25) of the smartphone (20), and encryption information is shared between the blood pressure monitor (10) and the smartphone (20) when the specific operation is performed within a predetermined time after the operation information is transmitted from the smartphone (20) to the blood pressure monitor (10).

### Advantageous Effects of the Invention

According to the present invention, pairing between an information terminal and a measurement device can be performed easily and safely.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a schematic diagram illustrating a schematic configuration of a management system 100;

FIG. 2 is a sequence chart illustrating a procedure of processing when pairing between a blood pressure monitor 10 and a smartphone 20 is successful;

FIG. 3 is a sequence chart illustrating a procedure of processing when pairing between the blood pressure monitor 10 and the smartphone 20 fails; and,

FIG. 4 is a sequence chart illustrating a procedure of processing when pairing between an activity amount meter and the smartphone 20 is successful.

### DESCRIPTION OF EMBODIMENTS

### Overview of Measurement Device and Information Terminal of Present Embodiment

The measurement device and the information terminal according to the present embodiment can communicate with each other by near-field wireless communication. In a state where the communication connection between the information terminal and the measurement device by the near-field wireless communication is established, the information terminal transmits, to the measurement device, operation information indicating a specific operation (for example, an operation for measuring a measurement target amount) for operating a sensor included in the measurement device, and outputs information for instructing the specific operation by voice, display, or the like. When the user performs the specific operation on the measurement device in accordance with the output information, the measurement device and the information terminal share encryption information for performing encrypted communication by the near-field wireless communication, and pairing is completed. The user can complete pairing between the measurement device and the information terminal only by performing an operation for establishing a communication connection between the measurement device and the information terminal (for example, an operation such as pressing a communication button provided on the measurement device) and a specific operation on the measurement device. Accordingly, it is possible to easily perform pairing between the measurement device and the information terminal.

Hereinafter, a configuration example of a management system 100 including a measurement device and an information terminal of the present embodiment will be described.

### System Configuration

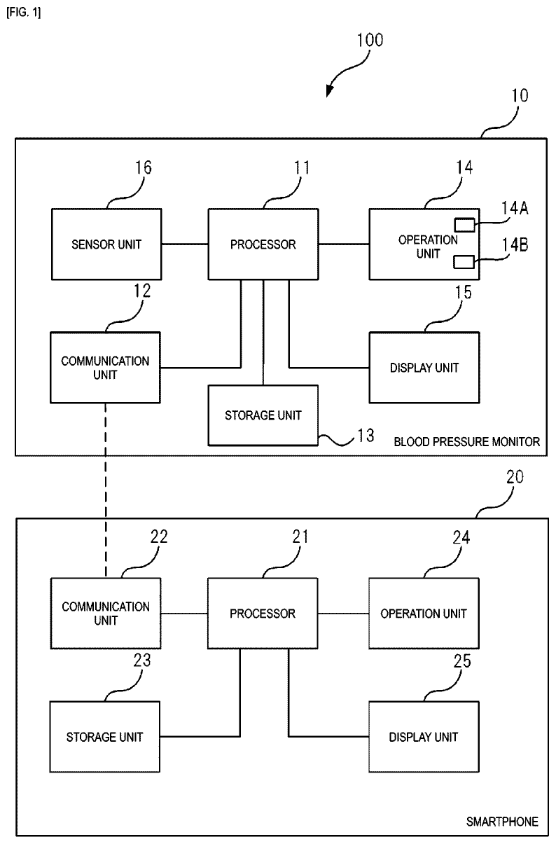

FIG. 1 is a schematic diagram illustrating a schematic configuration of the management system 100. The management system 100 includes a blood pressure monitor 10, which is an example of the measurement device, and a smartphone 20, which is an example of the information terminal, and is a system for managing measurement data, a recording medium, and the like of the blood pressure monitor 10 using the smartphone 20. The blood pressure monitor 10 and the smartphone 20 are configured to be able to communicate with each other by the near-field wireless communication. A method of the near-field wireless communication is not particularly limited, and for example, a method such as Wi-Fi, ANT, Bluetooth (trade name), or infrared communication can be used. In the following description, it is assumed that the near-field wireless communication method is Bluetooth (trade name). Hereinafter, an owner who owns both the blood pressure monitor 10 and the smartphone 20 is referred to as a user.

### Measurement Device

The blood pressure monitor 10 includes a processor 11, a communication unit 12, a storage unit 13, an operation unit 14, a display unit 15, and a sensor unit 16.

The sensor unit 16 includes a pressure sensor disposed in a cuff portion of the blood pressure monitor 10 as a measurement sensor, and detects, by the pressure sensor, a pulse wave from a blood vessel of the user under an appropriate cuff pressure. The blood pressure monitor 10 can calculate the blood pressure information including the maximum blood pressure, the minimum blood pressure, and the pulse based on the pulse wave detected by the sensor unit 16.

The communication unit 12 is a communication interface for performing the near-field wireless communication, and includes a communication antenna and various circuits.

The storage unit 13 includes a non-transitory storage medium such as a flash memory in addition to a work memory such as a random access memory (RAM). Various types of information such as measured blood pressure information are stored in this storage medium.

The operation unit 14 is an input unit such as a button or a touch panel that receives an input from the user, and receives various operations such as ON/OFF of a power supply, start of measurement of blood pressure information, and selection of an item from the user. The operation unit 14 includes a measurement start button 14A for instructing to start measuring the blood pressure information, and a communication button 14B for operating the communication unit 12 (enabling the near-field wireless communication). The measurement start button 14A and the communication button 14B may be hardware buttons, or may be software buttons displayed on the display unit 15 equipped with a touch panel.

The display unit 15 includes, for example, an organic electro-luminescence (EL) display, a liquid crystal display, or the like, and displays the measured blood pressure information and the like.

The processor 11 comprehensively controls each unit of the blood pressure monitor 10. The processor 11 is, for example, a central processing unit (CPU) that is a general-purpose processor that executes software (recording medium) to perform various functions, a programmable logic device (PLD) that is a processor whose circuit configuration can be changed after manufacturing, such as a field programmable gate array (FPGA), or a dedicated electric circuit that is a processor having a circuit configuration dedicatedly designed to execute specific processing, such as an application specific integrated circuit (ASIC). The processor 11 may be configured by one processor, or may be configured by a combination of two or more processors of the same type or different types (for example, a plurality of FPGAs or a combination of a CPU and an FPGA). For example, the processor 11 may include a processor that controls communication using the communication unit 12 and a processor that performs various types of control other than the communication. More specifically, the hardware structure of the processor 11 is an electric circuit (circuitry) in which circuit elements such as semiconductor elements are combined.

In detecting pressing of the measurement start button 14A included in the operation unit 14, the processor 11 receives an instruction to start measuring, pressurizes the cuff, and calculates the blood pressure information based on the pulse waves detected by the sensor unit 16 under an appropriate pressure of the cuff. Then, the processor 11 displays the calculated blood pressure information on the display unit 15. The processor 11 controls each component of the blood pressure monitor 10 so as to execute processing according to a user's operation performed via the operation unit 14.

In detecting the pressing of the communication button 14B included in the operation unit 14, the processor 11 activates the communication unit 12. It is preferable that the pressing operation of the communication button 14B necessary for activating the communication unit 12 is a simple operation so that even a user who is not good at operating the instrument can perform the pressing operation. To be specific, it is preferable that the communication unit 12 is activated by pressing the communication button 14B once in a short time (performing a so-called short-press operation) instead of continuously pressing the communication button 14B for a predetermined time (performing a so-called long-press operation).

In reference to FIG. 1, the blood pressure monitor 10 is illustrated as an example of the measurement device, but the blood pressure monitor 10 can be replaced with a weight scale, a body composition meter, a pulse rate meter, a heart rate meter, a thermometer, a blood glucose meter, a pulse oximeter, an activity amount meter, or the like. In any of these measurement devices, the sensor unit 16 includes various measurement sensors (a pressure sensor, a pulse wave sensor, a blood glucose sensor, a photoelectric sensor, a temperature sensor, an acceleration sensor, or the like) for measuring a physical quantity of a measurement target. In a case where the measurement device is a biological information measurement device, when the processor 11 detects pressing of a measurement start button 14A included in the operation unit 14, the processor 11 operates a measurement sensor (a pressure sensor, a pulse wave sensor, a blood glucose sensor, a photoelectric sensor, a temperature sensor, or the like) included in the sensor unit 16 to measure biological information. In a case where the measurement device is an activity amount measurement device, when the activity amount measurement device is moved, information corresponding to the movement is output from a measurement sensor (an acceleration sensor, an angular velocity sensor, or the like) included in the sensor unit 16.

In the biological information measurement device, an operation of pressing the measurement start button 14A (in other words, an operation for measuring biological information) constitutes a specific operation for operating the measurement sensor included in the biological information measurement device. In the activity amount measurement device, an operation of moving the activity amount measurement device constitutes a specific operation for operating the measurement sensor included in the activity amount measurement device. Operating the measurement sensor means starting the measurement when the measurement sensor has not started the measurement, and means changing the output of the measurement sensor when the measurement sensor is performing the measurement.

### Information Terminal

The smartphone 20 includes a processor 21, a communication unit 22, a storage unit 23, an operation unit 24, and a display unit 25.

The communication unit 22 is a communication interface for performing the near-field wireless communication, and includes a communication antenna and various circuits.

The storage unit 23 includes a non-transitory storage medium such as a flash memory in addition to a work memory such as a RAM. The storage medium stores various types of information including an application recording medium (an operation recording medium of the information terminal).

The operation unit 24 is an input unit such as a button or a touch panel that receives an input from a user, and receives various operations from the user.

The display unit 25 includes, for example, an organic EL display, a liquid crystal display, or the like.

The processor 21 comprehensively controls each unit of the smartphone 20. Similarly to the processor 11, the processor 21 includes one or a plurality of processors. The processor 21 executes various programs stored in the storage unit 23 to exhibit functions according to the programs.

### Method for Pairing Blood Pressure Monitor and Smartphone

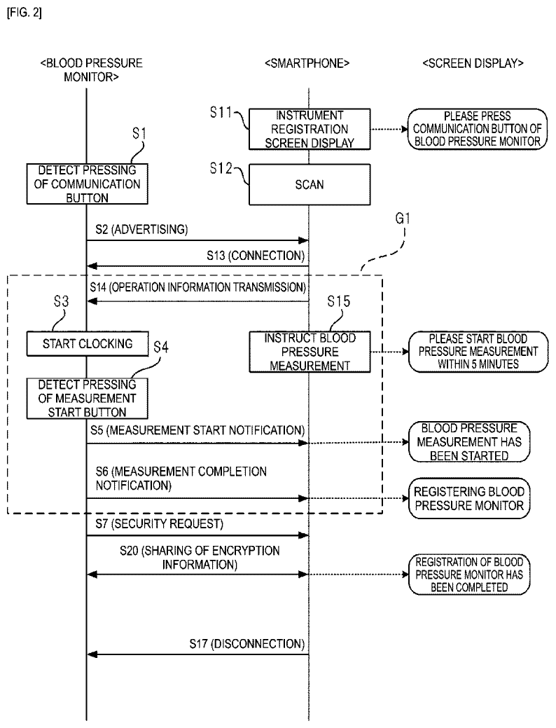

Next, a method for pairing the blood pressure monitor 10 and the smartphone 20 will be described. FIG. 2 is a sequence chart illustrating a procedure of processing when pairing between the blood pressure monitor 10 and the smartphone 20 is successful. Examples of messages each displayed on the display unit 25 of the smartphone 20 are illustrated at the right end of FIG. 2. It is assumed that a management application for managing the blood pressure information is installed and stored in advance in the storage unit 23 of the smartphone 20.

The user operates the smartphone 20 to activate the management application. When the management application is activated, the processor 21 of the smartphone 20 causes the display unit 25 to display an instrument registration screen (step S11). The processor 21 causes the communication unit 22 to start scanning (step S12). Scan means that the communication unit 22 enters a reception state and acquires information of surrounding devices.

As illustrated in FIG. 2, the instrument registration screen displayed in step S11 includes a message such as “Please press the communication button of the blood pressure monitor”. When the user confirms this message, the user prepares the blood pressure monitor 10 to be paired with the smartphone 20, turns on the power of the blood pressure monitor 10, and then presses the communication button 14B included in the operation unit 14 of the blood pressure monitor 10.

When the processor 11 of the blood pressure monitor 10 detects pressing of the communication button 14B (step S1), the processor 11 activates the communication unit 12 and causes the communication unit 12 to start advertising (step S2). Advertising refers to periodically transmitting a packet on an advertising channel.

When the processor 21 of the smartphone 20 acquires the packet transmitted from the blood pressure monitor 10 through the advertising channel, the processor 21 issues a connection request to the blood pressure monitor 10 via the communication unit 22 (step S13). When the processor 11 of the blood pressure monitor 10 responds to the connection request via the communication unit 12, a communication connection by the near-field wireless communication is established between the blood pressure monitor 10 and the smartphone 20.

When the communication connection with the blood pressure monitor 10 is established, the processor 21 of the smartphone 20 transmits operation information indicating a specific operation of the blood pressure monitor 10 (an operation of measuring blood pressure information, in other words, pressing of the measurement start button 14A) to the blood pressure monitor 10 via the communication unit 22 (step S14). The operation information is information for designating execution of a specific operation as a condition of authentication for starting pairing, and specifically includes the detail of the specific operation and an upper limit time (hereinafter, 5 minutes as an example) of a waiting time until the specific operation is executed. The processor 21 of the smartphone 20 causes the display unit 25 to display information instructing a specific operation included in the operation information (for example, a message instructing execution of a specific operation) (step S15). This message may be output by voice from a speaker, for example, instead of being displayed on the display unit 25. In step S15, for example, a message such as “Please start blood pressure measurement within 5 minutes (=upper limit time) from now” is displayed on the display unit 25. Note that the execution order of step S14 and step S15 may be reversed, or step S14 and step S15 may be performed in parallel.

When the operation information transmitted from the smartphone 20 by the processing of step S14 is received by the communication unit 12, the processor 11 of the blood pressure monitor 10 starts clocking in order to determine whether or not the specific operation included in the operation information has been performed until the upper limit time (5 minutes) included in the operation information elapses (step S3). Note that the operation information may not include the upper limit time. In this case, the upper limit time is stored in advance in the storage unit 13 of the blood pressure monitor 10, and the processor 11 of the blood pressure monitor 10 is configured to start clocking when the operation information is received.

In accordance with the message displayed on the display unit 25 of the smartphone 20 in the processing of step S15, the user attaches the cuff of the blood pressure monitor 10 to the arm or wrist, and then presses the measurement start button 14A to start measuring the blood pressure information.

After step S3, when the processor 11 of the blood pressure monitor 10 detects pressing of the measurement start button 14A before the upper limit time (5 minutes) included in the operation information (or stored in the storage unit 13) elapses (step S4), the processor 11 transmits a notification indicating that the measuring of the blood pressure information has been started to the smartphone 20 via the communication unit 12 (step S5). The processor 21 of the smartphone 20 that has received this notification causes the display unit 25 to display a message such as “Blood pressure measurement has been started”, for example. The processing of displaying the message is not essential and may be omitted. By displaying such a message, it becomes easy to recognize that the cooperation between the blood pressure monitor 10 and the smartphone 20 is successful, so that it is possible to allow the user to proceed with the work without anxiety.

After step S5, the processor 11 of the blood pressure monitor 10 calculates the blood pressure information based on the output of the sensor unit 16, causes the display unit 15 to display the blood pressure information, and ends the measurement of the blood pressure information. Then, the processor 11 transmits a notification indicating that the measurement of the blood pressure information has been completed to the smartphone 20 via the communication unit 12 (step S6), and further transmits a security request for requesting the start of pairing to the smartphone 20 via the communication unit 12 (step S7). The processor 21 of the smartphone 20 that has received the notification of step S6 causes the display unit 25 to display a message indicating that pairing is being performed, such as “Registering the blood pressure monitor”. The processing of displaying the message is not essential and may be omitted. By displaying such a message, it is possible to give a sense of security to the user.

By receiving the notification of step S6, the processor 21 of the smartphone 20 determines that the mutual authentication between the blood pressure monitor 10 and the smartphone 20 has been completed, and shares encryption information for performing encrypted communication by the near-field wireless communication with the blood pressure monitor 10 in response to the security request of step S7 (step S20). After step S7, the processor 11 of the blood pressure monitor 10 waits for a response from the smartphone 20, and shares the encryption information with the smartphone 20 in response to the response (step S20). Sharing of the encryption information means that the processor 11 of the blood pressure monitor 10 generates the encryption information, stores the encryption information in the storage unit 13, and transmits the encryption information to the smartphone 20, and the smartphone 20 stores the encryption information in the storage unit 23. The processor 21 of the smartphone 20 may generate the encryption information, store the encryption information in the storage unit 23, and transmit the encryption information to the blood pressure monitor 10, and the blood pressure monitor 10 may store the encryption information in the storage unit 13 to share the encryption information.

When the encryption information is shared between the blood pressure monitor 10 and the smartphone 20 by the processing of step S20, the processor 21 of the smartphone 20 causes the display unit 25 to display a message indicating that pairing has been completed, such as “Registration of the blood pressure monitor has been completed”.

Thereafter, the processor 21 of the smartphone 20 disconnects the communication connection with the blood pressure monitor 10 (step S17). Thereafter, secure communication using the encryption information becomes possible between the smartphone 20 and the blood pressure monitor 10.

Note that the processor 11 of the blood pressure monitor 10 may omit the notification of step S6 and issue a security request of step S7 together with the notification of step S5. In this case, by receiving the notification of step S5, the processor 21 of the smartphone 20 determines that the mutual authentication has been completed and starts the processing of step S20 in response to the security request of step S7.

Further, the detail of the specific operation included in the operation information transmitted in step S14 is not limited to “Measurement of blood pressure information” and may be “Pressing of the measurement start button 14A”. In this case, as described above, the processor 11 of the blood pressure monitor 10 omits the notification of step S6 and issues a security request of step S7 together with the notification of step S5.

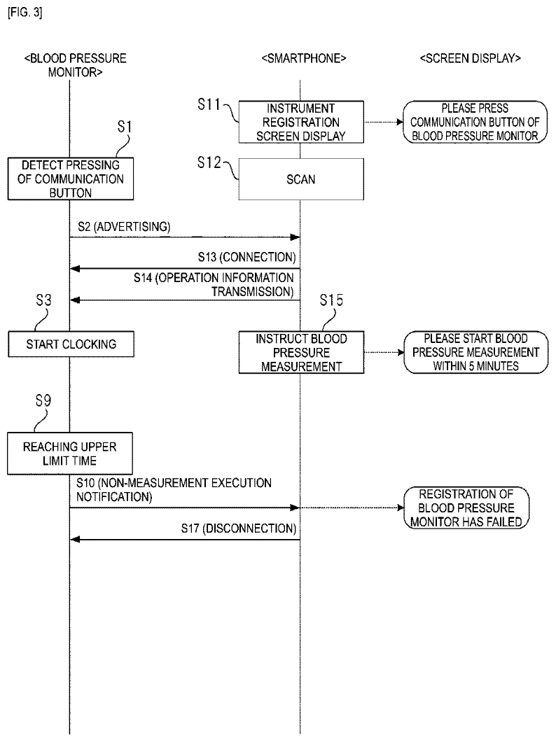

FIG. 3 is a sequence chart illustrating a procedure of processing when pairing between the blood pressure monitor 10 and the smartphone 20 fails. In the sequence chart illustrated in FIG. 3, the processing up to step S3 in the blood pressure monitor 10 is the same as that in FIG. 2, and the processing up to step S15 in the smartphone 20 is the same as that in FIG. 2.

When the upper limit time included in the operation information received from the smartphone 20 elapses without detecting the pressing of the measurement start button 14A after the clocking is started in step S3 (step S9), the processor 11 of the blood pressure monitor 10 transmits a notification indicating that the specific operation is not performed to the smartphone 20 via the communication unit 12 (step S10). Upon receiving this notification, the processor 21 of the smartphone 20 recognizes that the mutual authentication between the blood pressure monitor 10 and the smartphone 20 has failed, and causes the display unit 25 to display a message indicating that the pairing has failed, such as “Registration of blood pressure monitor has failed”, for example. Thereafter, the processor 21 disconnects the communication connection with the blood pressure monitor 10 (step S17).

As described above, in the management system 100, when the processor 11 of the blood pressure monitor 10 receives the operation information from the smartphone 20 and then determines that the specific operation included in the operation information is performed within the upper limit time included in the operation information (when pressing of the measurement start button 14A is detected at step S4), the processor 11 starts pairing with the smartphone 20. After transmitting the operation information to the blood pressure monitor 10 at step S14, the processor 21 of the smartphone 20 receives the notification of step S5 or step S6 from the blood pressure monitor 10 until the upper limit time included in the operation information elapses and starts pairing with the blood pressure monitor 10 when the processor 21 determines that the specific operation has been performed.

As described above, according to the management system 100, the user can establish a connection between the blood pressure monitor 10 and the smartphone 20 by pressing the communication button 14B of the blood pressure monitor 10, and in this state, pair the blood pressure monitor 10 and the smartphone 20 only by following a message (a message instructing execution of measurement of blood pressure information) output from the smartphone 20 that the user is accustomed to use and operating the blood pressure monitor 10 in accordance with the normal purpose of use. In this manner, even if the user is not familiar with the blood pressure monitor 10, pairing with the smartphone 20 can be completed without hesitation. In addition, the pairing is not completed unless the specific operation is performed within a predetermined time (the upper limit time included in the operation information) after the message instructing the execution of the specific operation is displayed on the smartphone 20. Therefore, it is possible to prevent pairing from being completed with an unintended combination of the blood pressure monitor 10 and the smartphone 20. In this manner, it is possible to secure the safety of the near-field wireless communication while making the pairing procedure easy for the user to understand.

Note that the processor 21 of the smartphone 20 may determine whether or not the specific operation has been performed within the upper limit time after the transmission of the operation information without waiting for the notification from the blood pressure monitor 10. For example, the processor 21 may turn on the microphone mounted on the smartphone 20 after step S14, and determine that the specific operation has been performed when the driving sound of the blood pressure monitor 10 (the driving sound of the pump generated when the cuff is pressurized) is detected by the microphone within the upper limit time. In this case, the processor 21 recognizes that the blood pressure monitor 10 and the smartphone 20 have been mutually authenticated by detecting the driving sound, and starts the processing of step S20. In the blood pressure monitor 10, when the pressing of the measurement start button 14A is detected, the processor 11 shifts the processing to step S20 and starts pairing with the smartphone 20.

In addition, in the management system 100, the processing surrounded by the broken line frame G1 in FIG. 2 may be continuously performed a plurality of times, and when the notification of step S6 is performed in each of the plurality of times (that is, when the specific operation is performed a plurality of times), the processing of step S20 may be performed. In this manner, it is possible to securely prevent pairing from being completed with an unintended combination of the blood pressure monitor 10 and the smartphone 20.

In order to enhance security, the following processing may be further performed in step S20. The processor 21 of the smartphone 20 causes the display unit 25 to display a message such as “Please input the measured blood pressure information” or “Please photograph the measurement result displayed on the blood pressure monitor 10 with the smartphone 20”. In accordance with this message, the user inputs the blood pressure information measured by the blood pressure monitor 10 to the smartphone 20 or captures an image of the blood pressure information using the smartphone 20. The processor 21 of the smartphone 20 acquires blood pressure information input by the user or blood pressure information recognized from a captured image, generates a hash value from the blood pressure information, and transmits the hash value to the blood pressure monitor 10 via the communication unit 22. When the hash value transmitted from the smartphone 20 matches the hash value generated from the blood pressure information measured by the blood pressure monitor 10, the processor 11 of the blood pressure monitor 10 shares the encryption information in step S20.

In the processing illustrated in FIG. 2, after step S5, the processor 11 of the blood pressure monitor 10 may disconnect from the smartphone 20, operate the sensor unit 16, calculate the blood pressure information based on the output of the sensor unit 16, display the blood pressure information on the display unit 15, and end the measurement of the blood pressure information. In this manner, it is possible to prevent the radio wave of the near-field wireless communication from affecting the blood pressure measurement. In this case, when the measurement of the blood pressure information ends, the processor 11 starts advertising, and establishes a connection with the smartphone 20 in response to a connection request from the smartphone 20 that has received the advertising. If the smartphone 20 with which the connection has been established matches the smartphone 20 that has transmitted the operation information in step S14, the processor 11 may perform the processing of step S6 and the processing of step S7 on the smartphone 20.

### Method for Pairing Activity Amount Meter and Smartphone

Next, a method for pairing the activity amount meter and the smartphone 20 when the blood pressure monitor 10 is replaced with the activity amount meter in the management system 100 will be described. Since the block configuration of the activity amount meter is the same as that of the blood pressure monitor 10, the following description will be made using the same reference numerals.

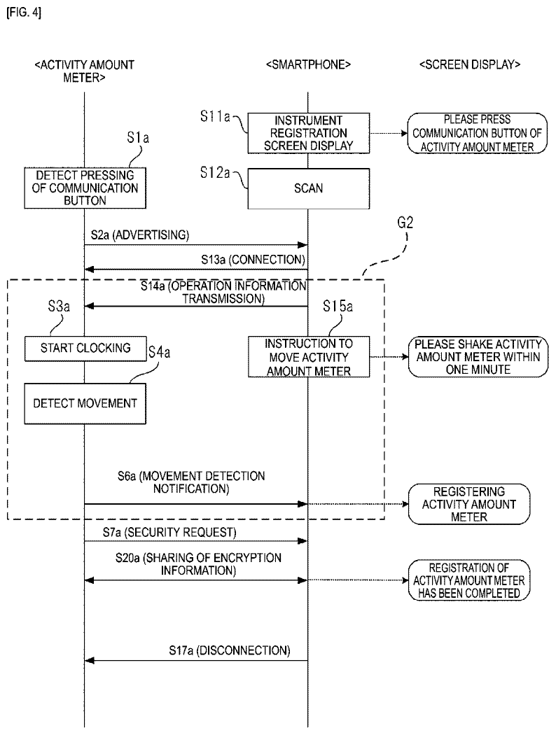

FIG. 4 is a sequence chart illustrating a procedure of processing when pairing between the activity amount meter and the smartphone 20 is successful. Examples of messages each displayed on the display unit 25 of the smartphone 20 are illustrated at the right end of FIG. 4. It is assumed that a management application for managing the activity amount of the user measured by the activity amount meter is installed and stored in advance in the storage unit 23 of the smartphone 20.

The user operates the smartphone 20 to activate the management application. When the management application is activated, the processor 21 of the smartphone 20 causes the display unit 25 to display an instrument registration screen (step S11a). The processor 21 causes the communication unit 22 to start scanning (step S12a).

As illustrated in FIG. 4, the instrument registration screen displayed in step S11a includes, for example, a message such as “Please press the communication button of the activity amount meter”. When the user confirms this message, the user prepares an activity amount meter to be paired with the smartphone 20, turns on the power of the activity amount meter, and presses the communication button 14B included in the operation unit 14 of the activity amount meter. The communication button 14B of the activity amount meter may also serve as a button for giving another instruction.

When the processor 11 of the activity amount meter detects pressing of the communication button 14B (step S1a), the processor 11 activates the communication unit 12 and causes the communication unit 12 to start advertising (step S2a).

When the processor 21 of the smartphone 20 acquires the packet transmitted from the activity amount meter through the advertising channel, the processor 21 issues a connection request to the activity amount meter via the communication unit 22 (step S13a). When the processor 11 of the activity amount meter responds to the connection request via the communication unit 12, a communication connection by the near-field wireless communication is established between the activity amount meter and the smartphone 20.

When the communication connection with the activity amount meter is established, the processor 21 of the smartphone 20 transmits operation information indicating a specific operation on the activity amount meter (an operation of moving the activity amount meter) to the activity amount meter via the communication unit 22 (step S14a). Specifically, the operation information includes the detail of the specific operation and the upper limit time (hereinafter, one minute as an example) of the waiting time until the specific operation is executed. The processor 21 of the smartphone 20 causes the display unit 25 to display information instructing a specific operation included in the operation information (for example, a message instructing execution of a specific operation) (step S15a). In step S15a, for example, a message such as “Please shake the activity amount meter up and down (or left and right) within one minute (=upper limit value) from now” is displayed on the display unit 25. Note that the execution order of step S14a and step S15a may be reversed, or step S14a and step S15a may be performed in parallel.

When the operation information transmitted from the smartphone 20 by the processing of step S14a is received by the communication unit 12, the processor 11 of the activity amount meter starts clocking in order to determine whether or not the specific operation included in the operation information has been performed until the upper limit time (1 minute) included in the operation information elapses (step S3a).

The user holds the activity amount meter in his/her hand and swings the hand to move the activity amount meter in accordance with the message displayed on the display unit 25 of the smartphone 20 in the processing of step S15a.

When the processor 11 of the activity amount meter detects a move (a vertical or horizontal motion) of the activity amount meter from the output of the acceleration sensor included in the sensor unit 16 before the upper limit time (one minute) included in the operation information elapses after step S3a (step S4a), the processor 11 transmits a notification indicating that the specific operation has been performed to the smartphone 20 via the communication unit 12 (step S6a). The processor 11 of the activity amount meter further transmits a security request for requesting to start pairing to the smartphone 20 via the communication unit 12 (step S7a). The processor 21 of the smartphone 20 that has received the notification of step S6a causes the display unit 25 to display a message indicating that pairing is being performed, such as “Registering the activity amount meter”, for example. The processing of displaying the message is not essential and may be omitted.

By receiving the notification of step S6a, the processor 21 of the smartphone 20 determines that the mutual authentication between the activity amount meter and the smartphone 20 is completed, and shares the encryption information with the activity amount meter in response to the security request of step S7a (step S20a). After step S7a, the processor 11 of the activity amount meter waits for a response from the smartphone 20, and shares the encryption information with the smartphone 20 in response to the response (step S20a).

After the processing of step S20a, the processor 21 of the smartphone 20 causes the display unit 25 to display a message indicating that pairing has been completed, such as “Registration of the activity amount meter has been completed”.

The processor 21 of the smartphone 20 then disconnects the communication connection with the activity amount meter (step S17a). Thereafter, secure communication using the encryption information can be performed between the smartphone 20 and the activity amount meter.

When the upper limit time included in the operation information received from the smartphone 20 elapses without detecting the movement of the activity amount meter after the clocking is started at step S3a, the processor 11 of the activity amount meter transmits a notification indicating that the specific operation has not been performed to the smartphone 20 via the communication unit 12. Upon receiving this notification, the processor 21 of the smartphone 20 recognizes that the mutual authentication between the activity amount meter and the smartphone 20 has failed, and causes the display unit 25 to display a message indicating that the pairing has failed, such as “Registration of activity amount meter has failed”, for example. Thereafter, the processor 21 disconnects the communication connection with the activity amount meter.

In this manner, the user can establish the connection between the activity amount meter and the smartphone 20 by pressing the communication button 14B of the activity amount meter, and in this state, complete the pairing of the activity amount meter and the smartphone 20 only by moving the activity amount meter in accordance with the message output from the smartphone 20 that the user is accustomed to use. In this manner, even an activity amount meter with which the user is not familiar can complete pairing with the smartphone 20 without hesitation.

The specific operation included in the operation information transmitted to the activity amount meter in step S14a of FIG. 4 may be an operation of moving the activity amount meter in a specific direction, rather than an operation of simply moving the activity amount meter. For example, the specific operation may be an operation of moving the activity amount meter up and down, an operation of moving the activity amount meter left and right, or the like. In this case, when the processor 11 of the activity amount meter detects that the activity amount meter is moving according to the specific operation after step S3a, the processor 11 performs the processing of step S6a and thereafter. In this manner, it is possible to more accurately perform mutual authentication between the activity amount meter and the smartphone 20, and it is possible to securely prevent pairing from being completed with an unintended combination of the activity amount meter and the smartphone.

The processor 21 of the smartphone 20 may give an instruction to place the activity amount meter on the smartphone 20 instead of giving an instruction to move the activity amount meter at step S15a. After giving this instruction, the processor 21 operates the vibration motor mounted on the smartphone 20. When the vibration motor of the smartphone 20 operates, the activity amount meter placed on the smartphone 20 also vibrates, and thus the processor 11 of the activity amount meter detects the movement of the activity amount meter at step S4a. As a result, the processing of step S6a and the processing of step S7a are performed, and the pairing is completed in step S20a.

Also, in the sequence chart of FIG. 4, the processing surrounded by the broken line frame G2 may be continuously performed a plurality of times, and the processing of step S20a may be performed when the notification of step S6a is made in each of the plurality of times (that is, when the specific operation is performed a plurality of times). In this manner, it is possible to securely prevent pairing from being completed with an unintended combination of the activity amount meter and the smartphone. In addition, the plurality of specific operations may all have the same operation detail, but it is preferable that the plurality of specific operations have different operation details (two types of operations such as an operation of moving up and down and an operation of moving left and right, or two types of operations such as an operation of moving and an operation of staying).

Although various embodiments have been described above, it is needless to say that the present invention is not limited to such examples. It will be apparent to those skilled in the art that various changes and modifications can be made within the scope of the claims, and it is understood that these are naturally belong within the technical scope of the present invention. Further, components of the above-described embodiments may be combined as desired within a range that does not depart from the spirit of the present invention.

### REFERENCE NUMERALS LIST

G1, G2 Broken line frame; 10 Blood pressure monitor; 11, 21 Processor; 12, 22 Communication unit; 13, 23 Storage unit; 14A Measurement start button; 14B Communication button; 14, 24 Operation unit; 15, 25 Display unit; 16 Sensor unit; 20 Smartphone; 100 Management system.

What is claimed is:

1. A measurement device comprising:
a near-field wireless communication unit;
a processor; and
a measurement sensor, wherein
the processor
receives, in a state in which a communication connection with an information terminal by near-field wireless communication is established, operation information indicating a specific operation for operating the measurement sensor from the information terminal; and
shares, with the information terminal and in a case where the specific operation is determined to be performed within a predetermined time after reception of the operation information, encryption information for performing encrypted communication by the near-field wireless communication.

2. The measurement device according to claim 1, wherein
the measurement device measures biological information or an activity amount, and
the specific operation includes an operation of measuring the biological information or an operation of moving the measurement device.

3. The measurement device according to claim 1, wherein
the processor receives the operation information from the information terminal at a plurality of timings, and shares the encryption information with the information terminal in a case where the specific operation is determined to be performed within a predetermined time after reception of each piece of the operation information.

4. The measurement device according to claim 3, wherein
a plurality of pieces of the operation information received at the plurality of timings include information indicating a first operation as the specific operation and information indicating a second operation as the specific operation, the second operation being different from the first operation.

5. The measurement device according to claim 4, wherein
the measurement device measures an activity amount,
the first operation is an operation of moving the measurement device, and
the second operation is an operation of staying the measurement device.

6. The measurement device according to claim 2, wherein
the processor receives the operation information from the information terminal at a plurality of timings, and shares the encryption information with the information terminal in a case where the specific operation is determined to be performed within a predetermined time after reception of each piece of the operation information.

7. The measurement device according to claim 6, wherein
a plurality of pieces of the operation information received at the plurality of timings include information indicating a first operation as the specific operation and information indicating a second operation as the specific operation, the second operation being different from the first operation.

8. The measurement device according to claim 7, wherein
the measurement device measures an activity amount,
the first operation is an operation of moving the measurement device, and
the second operation is an operation of staying the measurement device.

9. An information terminal comprising:
a near-field wireless communication unit; and
a processor, wherein
the processor
transmits, to a measurement device and in a state in which a communication connection with the measurement device by near-field wireless communication is established, operation information indicating a specific operation for operating a measurement sensor included in the measurement device and outputs information instructing the specific operation; and
shares, with the measurement device and in a case where the specific operation is determined to be performed within a predetermined time after transmission of the operation information, encryption information for performing encrypted communication by the near-field wireless communication.

10. An operation recording medium of an information terminal including a near-field wireless communication unit, the operation recording medium causing a processor to execute:
transmitting, to a measurement device and in a state in which a communication connection with the measurement device by near-field wireless communication is established, operation information indicating a specific operation for operating a measurement sensor included in the measurement device and outputting information instructing the specific operation; and
sharing, with the measurement device and in a case where the specific operation is determined to be performed within a predetermined time after transmission of the operation information, encryption information for performing encrypted communication by the near-field wireless communication.
